(19) United States
(12) Patent Application Publication
(10) Pub. No.: US 2003/0082200 A1
(43) Pub. Date: May 1, 2003

(54) 52 kda protein from coagulase negative staphylococci and fragments thereof

(76) Inventors: Asa Ljungh, Lund (SE); Frederik Lundberg, Lund (SE); Li Dai-Qing, Lund (SE)

Correspondence Address: BACON & THOMAS, PLLC, 625 SLATERS LANE, FOURTH FLOOR, ALEXANDRIA, VA, 22314

(21) Appl. No.: 10/203,613

(22) PCT Filed: Feb. 16, 2001
(86) PCT No.: PCT/SE01/00340

(30) Foreign Application Priority Data
Feb. 17, 2000 (SE) 0000514-0

(51) Int. Cl. A61K 39/02; C07H 21/04; C12P 21/02; C12N 1/21; C07K 14/315

(52) U.S. Cl. 424/190.1; 435/69.3; 435/320.1; 435/252.3; 530/350; 536/23.7

(57) ABSTRACT

A protein isolated from Staphylococcus epidermidis having an approximate MW of 52 kD determined by SDS-PAGE and an N-terminal amino acid sequence (SEQ ID NO:1), and antigenic determinant-containing fragments of the protein, optionally coupled to an inert carrier or matrix, are disclosed. Disclosed are also a recombinant DNA molecule coding for the protein or the protein fragments; a vector comprising the DNA molecule or the corresponding RNA molecule; antibodies or antigen-binding peptides recognizing and specifically binding to the protein or protein fragment; use of the protein or protein fragment, or the vector, for the production of vaccines against Staphylococcal infections; use of the antibodies or antigen-binding peptides for the production of a medicament for passive immunization; a vaccine against Staphylococcal infections comprising the protein or protein fragment, or the vector, a medicament for passive immunization comprising the antibodies or antigen-binding peptides; and a method of prophylactic and/or therapeutic treatment of Staphylococcal infections.

The present invention relates to a 52 kD protein isolated from Staphylococcus epidermidis and antigenic determinant-containing fragments of the protein. The protein binds to immobilized vitronectin (Vn). The invention also relates to applications of the protein or the fragments e.g. in eliciting antibodies, and vaccines for active immunization and medicaments for passive immunization.

### BACKGROUND OF THE INVENTION

Coagulase-negative staphylococci (CoNS) are considered as major pathogens of indwelling catheter and prosthetic device infections, thus contributing to the majority of hospital-acquired infections (1). The pathogenesis of these infections, as it is seen today, is that microbes adhere to the surface of biomaterial to which host factors (e.g. proteins and glycosaminoglycans) adsorb, slow down their metabolism and protect themselves from host defense or antibiotics by producing a so-called biofilm. Among host molecules, which have been proposed to mediate adhesion of bacteria are fibrinogen, fibronectin, vitronectin, collagen and thrombospondin (2-5).

The present invention is based on research work that focuses on cell wall structures in coagulase-negative staphylococci, which bind vitronectin.

Vitronectin is a glycoprotein with multiple functions in the host, cell attachment, inhibition of complement activation and regulation of blood coagulation (6). It is present in human connective tissue and in plasma and was recently detected in human cerebrospinal fluid (CSF) (7). It was traced to human plasma and its concentration in cerebrospinal fluid increased in the patients with broken blood-brain barrier. Vitronectin was shown to mediate adhesion of S. epiderniidis strains to polyvinylchloride exposed to cerebrospinal fluid in vitro (3).

Streptococci, Staphylococcus aureus, Escherichia coli, Helicobacterpylori, Pneumocystis carinji and Candida albicans were earlier shown to bind vitronectin in vitro (8-11). One 60 kDa surface protein from S. aurezis strain V8 recognized soluble and immobilized vitronectin. In spite of the fact that there are reports about coagulase-negative staphylococci binding vitronectin, the binding components have not been identified (3, 5).

Proteins binding to immobilized vitonectin or antigenic determinant-containing fragments of the proteins would be useful as immunizing components in the development of vaccines against Staphylococcal infections, and in the production of antibodies or antigen-binding peptides recognizing and specifically binding to the proteins or protein fragments for use in vaccines for passive immunization.

### DESCRIPTION OF THE INVENTION

The present invention provides a 52 kD protein that binds to immobilized vitronectin. The protein has been purified from a cell wall extract from the strain S. epidermidis BD5703 isolated from an infected cerebrospinal fluid shunt system, reported to bind vitronectin (3).

A first aspect of the invention is therefore directed to a protein isolated from Staphylococcus epidermidis having an approximate molecular weight (MW) of 52 kD determined by SDS-PAGE and an N-terminal amino acid sequence of

| Thr Ala Asp Pro Pro Ala Asp Lys Thr Ser | SEQ ID NO:1 |
|---|---|
| 1 5 10 | |

and antigenic determinant-containing fragments of the protein.

Antigenic determinants contained in the protein of the invention may be identified by any method known in the art, e.g. by sequencing antibody binding sites.

The antigenic determinant-containing fragments of the protein of the invention will comprise at least 5, preferably at least 10, and most preferably at least 15 amino acid residues to be sure that the antigenic determinant is present in the peptide fragment. These fragments may be used e.g. as probes, diagnostic antigens, and vaccine components, possibly coupled to carriers.

In a preferred embodiment of this aspect of the invention the protein or protein fragment according to the invention is coupled to an inert carrier or matrix. The carrier may be e.g. plastic surfaces, such as surface of implants, microplates, beads etc.; organic molecules such as biotin; proteins, such as bovine serum albumin; peptide linkers, polypeptides e.g. resulting in fusion proteins. The matrix may be particles used for chromatographic purposes, such as Sepharose®.

A second aspect of the invention is directed to a recombinant DNA molecule coding for a protein or a protein fragment according to the invention.

With the aid of sequencing techniques known in the art, the 52 kD protein of the invention may be sequenced, and with the knowledge of the amino acid sequence of the protein of the invention, it will be possible to deduce nucleotide sequences that encode the protein of the invention or fragments of the protein. Then, recombinant DNA molecules coding for the protein or a protein fragment according to the invention may be prepared.

A third aspect of the invention is directed to a vector selected from the group consisting of plasmids, phages or phagemides comprising a DNA molecule according to the invention or the corresponding RNA molecule.

These vectors may be used for the production of the protein or protein fragments of the invention. They may also be used in vaccines.

A fourth aspect of the invention is directed to antibodies or antigen-binding peptides recognizing and specifically binding to a protein or protein fragment according to the invention.

The antibody of the invention may be a monoclonal antibody or monospecific polyclonal antibody recognizing and specifically binding to an antigenic compound according to the invention, i. e. recognizing and specifically binding to the protein or antigenic determinant-containing fragments of the protein or the invention.

The antibody of the invention can be prepared by using immunization procedures well known in the art, or by well known methods based on recombinant technology making use of suitable host cells of eukaryotic or prokaryotic origin.

The specific binding of an antibody to an antigenic compound of the invention requires e.g an affinity constant of at least 10⁷ liters/mole, preferably at least 10⁹ liters/mole.

The antibodies and antigen-binding peptides of the invention may be used for diagnostic purposes, but preferably in vaccines for passive immunization.

The antigen-binding peptide recognizing and binding to an antigenic compound according to the invention may be the antigen-binding part of an antibody or a synthetic compound which mimics the three-dimensional structure of the antigen-binding part of an antibody.

The antibodies and antigen-binding peptides of the invention may be used for diagnostic purposes, but preferably in vaccines for passive immunization.

A fifth aspect of the invention is directed to the use of a protein or protein fragment according to the invention, optionally in immobilized form, as an immunizing component in the production of a vaccine against Staphylococcus infections.

A sixth aspect of the invention is directed to the use of a vector according to the invention for the production of a vaccine against Staphylococcal infections.

A seventh aspect of the invention is directed to the use of-antibodies or antigen-binding peptides according to the invention for the production of a medicament for the passive immunization of a mammal against Staphylococcus infections.

An eighth aspect of the invention is directed to a vaccine against Staphylococcal infections comprising as an immunizing component a protein or protein fragment according to the invention, optionally in immobilized form.

A ninth aspect of the invention is directed to a vaccine against Staphylococcal infections comprising a vector according to the invention.

A DNA molecule of the invention, or the corresponding RNA, may be used in a vector for vaccine purposes.

A tenth aspect of the invention is directed to a medicament for the passive immunization of a mammal, especially a human being, against Staphylococcus infections comprising antibodies or antigen-binding peptides according to the invention.

An embodiment of the invention comprises the passive immunization of patients with an impaired immune defense or patient awaiting major surgery, such as patients in line for an organ transplantation or awaiting the insertion of a prosthetic device, such as a hip prosthesis or similar major surgical intervention. According to the present invention, a high dose of antibodies, or antigen-binding peptides according to the invention, against the novel protein can be administered to any patient before or at the time of hospitalization, in order to prevent Staphylococcus infection.

The vaccines may contain other ingredients selected with regard to the intended administration rout, and these ingredients are chosen by the vaccine manufacturer in collaboration with pharmacologists. Examples of administration routs include intravenous administration, percutaneous administration, intramuscular administration, oral and nasal administration.

An eleventh aspect of the invention is directed to a method of prophylactic and/or therapeutic treatment of Staphylococcu infections in a mammal comprising administration to said mammal of an immunologically effective amount of a vaccine according to any one of the vaccines of the invention.

The present invention will now be further illustrated by reference to the following description of drawings, experiments and specific embodiments of the invention, which are not to be considered as limitations to the scope of the invention defined by the claims.

### DESCRIPTION OF THE DRAWINGS

FIG. 1 shows a diagram of binding of soluble ¹²⁵I-labeled vitronectin (Vn) (μg/ml) to immobilized S. epidermidis strain BD5703 on ELISA plate wells (◯) or only to the BSA coated wells (●). Duplicate samples were repeated twice. The data are presented as mean values.

FIG. 2 shows a diagram of adhesion of 6 staphylococcal strains to Vn coated wells (2 μg/ml). S. aureus strain V8 and S. epidermidis strain RP12 were used as positive and negative control respectively. * p<0.05 compare to S. aureus V8. Triplicate samples were made and repeated twice. Data are presented as mean values with SEM

### DESCRIPTION OF EXPERIMENTS

Materials and Methods

Human Plasma and Proteins

Fresh human plasma was purchased from the blood bank, University Hospital of Lund. Human vitronectin, fibronectin and hemopexin were purified as previously described (12-14). Human fibrinogen was purchased from Imco AB., Stockholm, Sweden. Human plasminogen and IgG were purchased from Sigma Chemical Co., St. Louis, USA. Human thrombospondin was a kind gift from Professor J Lawler, Boston, USA. Bacterial strains and culture conditions

S. epidermidis strains BD5703, BD12213 and BD14611 were isolated from patients with CSF shunt infections, Dept Clinical Bacteriology, Lund University Hospital, Lund. S. epideriitidis strain RP12 and S. haemolyticus strain SM131, isolated from patients with catheter-associated sepsis and osteomylitis respectively, served as reference strains (5). S. aureus strain V8 was used as positive control for Vn binding. Bacteria were cultured in Todd-Hewitt (TH) broth (Difco Laboratories, Detroit, USA) at 37° C. except when different media were compared. Cells were harvested during stationary growth phase (after 20-22 hours), washed twice in phosphate-buffered saline (PBS) at pH 7.2 by centrifugation (2800×g, 15 min), resuspended at a final density of 10¹⁰ cells/ml in PBS and used promptly for various binding assays.

Bacterial Binding of Soluble Vn

Fifty micrograms of Vn was labeled with Na ¹²⁵I (Amersham Plc., Buckinghamshire, UK) using the modified chloramine-T method (specificity, 1×10⁶ cpm/μg), and used in a minor modified soluble binding test with final reaction volume of 150 μl in PBS (15). During binding experiments, radiolabeled protein were diluted to approx. 0.025 μg (25,000cpm) and incubated with bacteria (10⁹ cells per test tube). In an alternative method, cells of BD5703 were immobilized on ELISA plates (Costar Co., Cambridge, USA) by incubating 100 μl of bacterial suspension (2×10⁷ cells in PBS) for 30 min at 37° C. Non-attached bacteria were removed by 3 washes with PBS. The remaining sites on the plastic were blocked by 2%, a bovine serum albumin in PBS (BSA-PBS) for 1 hour at 37° C. The wells were washed with PBS, and two-fold diluted ¹²⁵I-labeled Vn in 1% BSA-PBS (maximally 5 μg/ml) was added. Wells coated with 1% BSA served as background. After incubation at 37° C. for 45 min and washes with PBS, individual wells were removed and the radioactivity was measured in a gamma counter (LKB-Wallac, Turku, Finland).

Bacterial Binding of Immobilized Vn

Vn was immobilized on ELISA plates (0.2 μg/well). Binding was quantitated using bioluminescence. ATP Monitoring Reagent and ATP standard were from BioThema, Stochholm, Sweden. Quantitation of adherent bacterial cells was achieved by multiplying total added bacteria with the percentage from retained adenosine triphosphate (ATP) released by bound bacteria in relation to total added ATP produced by 100 μl bacteria suspension (1×10⁷ cells) (3). Wells coated only with 2% BSA-PBS served as background. The values of these were subtracted from the values of other wells in the experiment before the percentages of binding were calculated.

Heat Anidproteolytic Treatment and Inhibition Tests

³H-labeled bacteria were used for these tests. In brief, half a milliliter of an overnight cultured bacteria in TH broth was diluted in 5 ml TH broth containing 50 μCi of [methyl-³H]thymidine (Amersham Plc., Buckinghamshire, UJK) and cultured for 5 hours at 37° C. on a gyrator shaker with vigorous agitation.

3H-labeled bacteria cells (5×10⁸) were suspended in 1 ml PBS heated at 100° C. for 30 min and rapidly cooled. The same amount of bacteria was treated by pronase E, protease K and trypsin (Sigma Chemical Co., St. Louis, USA) as previously described (15). After washes in PBS, 100 μl (1×10⁷ cells) treated bacterial suspension were added into the Vn coated wells.

In inhibition tests, crude extract by 1M LiCl (5 μg/ml), crude extract treated by trypsin and dialyzed against PBS (5 μg/ml,), heparin (2 mg/ml), purified binding proteins (60 and 52 kDa) from Vn-affinity chromatography (2 μg/ml) and purified proteins (21 and 16 kDa) from Mono-S® column (2 μg/ml) were preincubated with Vn coated wells for 2 hours at 22° C., and then 100 μl ³H-labeled bacteria (1×10⁷cfu/ml) were added. In another experiment, 50 μl bacteria (2×10⁷cfu/ml) were mixed with the same volume of four mono-carbohydrates (0.1M D-fucose, 0.1M D-galactose, 0.1M D-glucose and 0.1M D-mannose), heparin (2 mg/ml), hyaluronic acid (2 mg/ml) or Vn (20 μg/ml). These mixtures with bacteria were incubated for 1 hour at 37° C. Heparin sodium salt and hyaluronic acid were purchased from Fluka Chemie AG, Buchs, Switzerland)

Finally, all wells were washed by PBS 5 times, individual wells were removed and immersed in 2.5 ml scintillation fluid (BDH chemicals, Pool, UK) and the radioactivity was counted in a liquid scintillation counter (Beckman Instruments, Fullerton, Calif., USA). Quantitation of relative binding percentage was achieved by the radioactivity from experimental groups in relation to the radioactivity from non-treated group.

Trypsin Treatniesit of Vn-coated Wells

The wells pre-adsorbed with Vn were trypsinized at a concentration of 2 μg/well in PBS with 1 mM CaCl₂ (37° C. for 2 hours) following an additional incubation with soybean trypsin inhibitor (4 μg/well) at 37° C. for 30 min in same buffer (15). The wells were then blocked by BSA and subjected to binding assay with ³H labeled bacteria.

SDS-PAGE, Autoradiograph Assay and Inhibition Tests

SDS-PAGE was performed on mini-Protean II cell (Bio-Rad, Richmond, Calif.) as previously described (16). The bacterial surface structures were extracted by 1M lithium chloride (pH 5) with protease inhibitor (Boehringer Mannheim, Mannheim, Germany) at 37° C. for 2 hours with end to end rotation. Extract was subjected to homogenous gel (12%) or gradient gel (4-15%) electrophoresis. The running and transfer conditions were as previously described (17).

The PDVF membranes (Micron Separations INC., Westborough, USA) were saturated with incubation in blocking buffers I and II (buffer I containing 0.6% ethanolamine, 0.9% glycine, 1% polyvinylpyrrolidone and 25% methanol pH 9.6; buffer II containing 0.6% ethanolamine, 0.9% glycine, 0.05% gelatin hydrolysate and 0.125% Tween 20 pH 9.6) for 15 min respectively (18). Saturated membranes were rinsed three times in PBST (PBS with 1% Tween 20). Then, the membranes were incubated with ¹²⁵I-labeled Vn (0.4 μg/ml) in PBST or in PBST supplemented with 1.05 M sodium chloride (final sodium chloride concentration 1.2M) overnight at 20° C. The membranes were washed and dried, then examined by autoradiography with X-Omat AR film (Eastman Kodak) after 3 days storage at −70° C.

For inhibition tests, the membrane with retained bacterial surface molecules was cut to individual strips, and then incubated with labeled Vn (0.4 μg/ml) in PBST mixed with different unlabeled competing molecules, including Vn (10 μg/ml), fibronectin (10 μg/ml), fibrinogen (10μg/ml), thrombospondin (0 μg/ml), plasminogen (10 μg/ml), human immunoglobulins (10 μg/ml), hemopexin (10 μg/ml), heparin (1 mg/ml). In a separate experiment, the strips were preincubated with heparin (1 mg/ml) and hyaluronic acid (1 mg/ml) for 1 hour at 20° C. prior to adding labeled Vn.

Periodate Treatment and Immunoblot

The PDVF strips with retained BD5703 surface proteins were treated by 10 mM sodium periodate (Sigma Chemical Co., St. Louis, USA) in 100 mM acetate buffer (pH 2.9) according to the method described by Bouchez-Mahiout et al in 1999 (19). After treatment, the strips were washed by acetate buffer with 0.05% Tween-20, and then by PBST. Vn (2 μg/ml) was treated in same procedure and dialyzed against PBS with three time changes. The strips were incubated with periodate treated Vn in PBST overnight at 4° C. with gentle shaking. After three washes by PBST, primary antibodies to Vn, which were raised in rabbits as earlier described, diluted 1:400 in washing buffer (20 mM Tris buffer, pH 8.6, containing 0.5% Gelatin hydrolysate, 0.1% Tween 20, 350 mM NaCi) were added for 2 hours at 20° C. The strips were washed three times again and incubated with peroxidase-conjugated swine anti-rabbit immunoglobulins (DAKO, Copenhagen, Denmark) diluted 1:2000 in washing buffer for further 2 hours. After repeated washing, bound materials were detected by incubation in 50 mM sodium acetate buffer (pH 5) containing 0.04% 3-amino-9-ethylcarbazole and 0.015% H₂O₂.

Isolation of Bacterial Proteins Recoginizing Vn

One and a half milligram of Vn was coupled to one milliliter HiTrap™ N-hydroxysuccinimide (NHS)-activated affinity column purchased from Pharmnacia, Uppsala, Sweden. The coupling procedure was according to the manufacturer's instruction. About 0.45 mg surface extract in 35 ml 10 mM Tris-HCl buffer (pH 7.5) containing 0.1 M LiCl with protease inhibitor was applied to the Vn-column which was equilibrated by the same buffer through a P-1 pump (Pharmacia, Uppsala) at a flow rate of 30 ml/hour, followed by 10 ml equilibration buffer for washing at 20° C. The column was eluted by a lithium chloride gradient (0.1-2M), and several fractions were collected according to their UV absorbency. The fractions were dialyzed against PBS (Dialysis tubing was from Spectrum Medical Industries, Inc., Houston, USA) and then subjected to 12% SDS-PAGE. Silver staining was done as described in Pharmacia bulletin (Science Tools 2, 2; 1997) Ion-exchange chromatography with the FPLC® system (Pharmacia, Uppsala) was performed on a Mono S® High Resolution 5/5 column (Pharmacia, Uppsala). The FPLC® system was equilibrated with 10 times diluted PBS (pH 7.2) (buffer A), then 1M sodium chloride in buffer A (buffer B) and buffer A again. Crude extract was dialyzed in dialysis tubing (cut off MW is 3500) against buffer A and concentrated to 400 μg/ml against polyethylene glycol 20000 purum (PEG). The sample was centrifuged at 10000 rpm 10 min in order to discard precipitation due to buffer exchange before it was injected into a Mono S® column using a 1 ml loop. A stable base line was established followed by washing using buffer A. The column was eluted by gradient buffer B (0-100%) during 60 min at 1 ml/ml flow rate. The fractions were collected and subjected to SDS-PAGE analysis and transferred onto PVDF membrane as described above. The Vn binding molecules were stained by Coomassie brilliant blue R-250 and detected by immunoblot assay as described above.

N-terminal Sequence Analysis for Vn Binding Proteins

Vn binding molecules were separated in 12% gel and transferred onto PVDF membrane as described above. Intensive distilled water washes were made after staining by Coomassie brilliant blue R-250. The blue bands were cut down and analyzed by an Applied Biosystem Prosice®.

Statistical Analyses

The F test was used prior to further statistical analyses. Paired one-tailed Student's t test and Mann-Witney U test were used when appropriate. A P value of <0.05 was considered to represent a significant difference.

Results

Binding of Bacteria to Vn in Fluid and Solid Phase

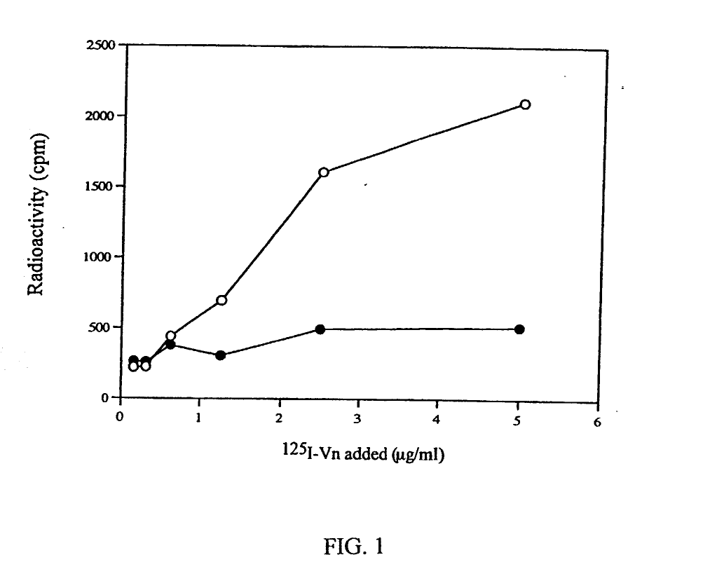

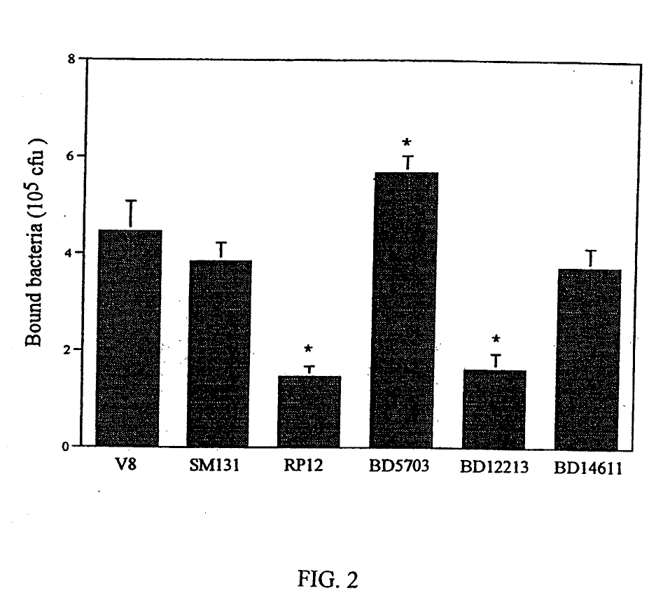

When coagulase-negative staphylococcal strains (SM131, RP12, BD5703, BD12213 and BD14611) were added to the solution of radioactively labeled Vn, the level of binding was substantially lower than of the reference strain V8 (14.2%). Only BD5703 bound more than 5% (less than 5% is considered as negative binding). The BD5703 binding was slightly influenced by culture conditions. Immobilized cells of S. epidermidis strain BD5703 bound ¹²⁵I-Vn in a dose-related manner (FIG. 1). S. epidermidis strains BD5703 and BD14611 bound immobilized Vn to a higher or similar extent compared to the positive control strains S. aureus V8 and S. haemolyticus SM131. Another S. epidennidis strain BD12213 bound to a lower extent (FIG. 2).

Heating, Protease Treatment and Replacement Tests

The binding of immobilized Vn by BD5703 and V8 decreased significantly by protease treatment. Heating did not influence the binding (Table 1). Almost 95% of the binding of immobilized Vn by BD5703 was reduced by incubation with LiCl extract of the same strain. This effect could be abolished when the BD5703 extract was treated by trypsin. Soluble Vn also inhibited the binding of immobilized Vn about 60%. BD5703 binding molecules eluted from Vn affinity chromatography decreased the binding level more than 50%. The 21 and 16 kDa proteins purified from ion-exchange chromatography reduced the bin ding by 28% and 19% respectively (Table 2). None of the monosaccharides inhibited the binding

The binding of immobilized Vn was reduced significantly when bacteria were added with heparin and hyaluronic acid at the same time. Heparin showed a more than 2-fold inhibitory ability compared to hyaluronic acid. However, preincubation of Vn coated wells with heparin increased the binding (Table 2).

Trypsin Treatment of Immobilized Vn

After trypsin treatment of immobilized Vn no binding was obtained. Vn binding protein detected by autoradiography and inhibition tests

The 60 kDa surface protein of S. aureis V8 reacted strongly with Vn. The low Vn binder S. epidermidis RP12 was shown to have different binding molecules (5). The binding structures depended on the culture conditions. Vn was recognized by RP12 surface protein that was around 16 kDa no matter which media was used, but 100 and 52 kDa binding proteins were only expressed when bacteria were cultured on blood agar. Expression of S. epidermidis BD5703 binding molecules was also influenced by culture conditions. Bacteria grown on blood agar or in TH broth produced 4-5 times more surface proteins compared with that from TSB broth. (Bio-Rad protein assay). There were two binding bands around 100 and 16 kDa when BD5703 was grown on blood agar, but the intensity of 100 kDa band was too weak to show (this band could be detected by immunoblot). Four proteins around 52, 38, 21 and 16 kDa were identified after bacteria were grown in TH and TS broths. When BD 12213 and BD14611 were cultured in TH broth both of them expressed the 21 kDa surface protein. BD1461 1 also expressed 38 and 16 kDa binding proteins. A higher sodium chloride concentration (1.2M) erased most of the binding bands, but 60 kDa from V8, 16 kDa from RP12 and 21 kDa from shunt infection isolates were still present. The binding proteins from BD5703 grown in TH broth were not influenced by the separation running condition (reduced or non-reduced condition).

The binding of labeled Vn to BD5703 surface proteins could be inhibited by unlabeled Vn. The binding of the 52 kDa, 38 and 16 kDa proteins were slightly blocked by fibronectin, fibrinogen, thrombospondin, hemopexin, plasminogen and IgG, but the 21 kDa protein was not as shown by autoradiography. Heparin could almost erase the 52, 38 and 16 kDa bands, but not 21 kDa. Hyaluronic acid also inhibited the 52 and 38 kba, but not the 21 and 16 kDa.

Periodate Treatment and Immuizoblot

After periodate treatment of bacterial surface structures, four proteins recognized periodate treated Vn. The molecular weights were 52, 38, 21 and 16 kDa.

Purification of Vn Binding Components

After the extract from BD5703 grown in TH broth was passed though the Vn affinity column, the column was washed using 30 ml gradient lithium chloride (0.1-2M). Five fractions were collected according to UV-monitor. SDS-PAGE and silver staining indicated three binding proteins with 60, 52 and 38 kDa molecular weights. Among these molecules, the 38 kDa protein was the first one to be eluted by LiCl and was difficult to separate from small proteins around it. The 60 kDa protein was eluted by 0.25-0.61M LiCl, and its major part was eluted by 0.34-0.43M LiCl. A third, the 52 kDa protein, was eluted by 0.340.7M LiCl, and its major part was eluted by 0.53-0.61M LiCl. These bands were recognized by Vn in immunoblot, and the antibody control (membrane incubated with anti-Vn antibody and secondary antibodies) indicated no leakage of coupled Vn. After the LiCl gradient, 0.1 M glycine-HCl (pH 3.0) was tried to elute high affinity binding proteins. No protein was eluted. Protein precipitation occurred during dialysis of BD5703 crude extract against 10 times diluted PBS. The pellet could be solubilised by sample buffer of SDS-PAGE (0.5M Tris-HCl containing 10% glycerol and 10% SDS, pH 6.8), and most of the proteins with molecular weights above 35 kDa were detected by SDS-PAGE and Coomassie brilliant blue R-250 staining in this solution. The supernatant of crude extract was subjected to Mono-S® column after the precipitate was removed, and eluted by a sodium chloride gradient. Twelve peaks were obtained from 0.23-0.61M sodium chloride gradient. SDS-PAGE, silver staining and immunblot assays were used to identify Vn binding proteins. The 21 and 16 kDa proteins were mainly in fraction 3 (around 0.3M NaCl) and 11 (around 0.6M NaCl) respectively (FIG. 7).

N-terminal Sequence Analysis

The N-terminals of binding proteins eluted from affinity column (60 and 52 kDa) and ion-exchange column (21 and 16 kDa) were sequenced. The 38 kDa was excluded since it was difficult to purify. The 60 kDa protein was blocked N-terminally. The 52, 21 and 16 kDa proteins were analyzed successfully and the sequences are shown in table 3. None of these three proteins had appreciable amino acid sequence homology with known Staphylococcal proteins (Microbial Genomes Blast Database).

### SUMMARY OF THE RESULTS

Adsorbed vitronectin (Vn) on the surface of implant may mediate bacterial adhesion. S. epidermidis strain BD5703 isolated from a cerebrospinal fluid shunt infection was tested for binding of immobilized Vn. It binds immobilized Vn to a higher extent than the positive control strain S. aureus V8. The binding could be inhibited by protease treatment, but not by carbohydrates (D-mannose, D-fucose, D-glucose and D-galactose). The binding was inhibited by its cell wall structures extracted by 1M lithium chloride, and this inhibitory effect was abolished when the extract was treated by trypsin. The binding was competed by soluble heparin, but not if Vn coated wells were preincubated with heparin. Vn binding proteins (VnBPs) of 52, 38, 21 and 16 kDa were detected by autoradiography when bacteria were grown in Todd-Hewitt broth. No band was influenced by periodate treatment, indicating that no glycosylation motif was involved in this interaction. The 52 and 38 kDa VnBPs and another 60 kDa binding protein were purified from Vn affinity chromatography by a lithium chloride gradient. The 21 and 16 kDa VnBPs were purified from Mono-S® ion-exchange column. All VnBPs could block bacterial binding of immobilized Vn to different extents except the 16 kDa. The N-terminal sequences of the 52, 21 and 16 kDa proteins were determined. No appreciable amino acid sequence homology in Staphylococcal protein database was found. The results demonstrates that ligand-receptor interaction may exist between S. epidermidis and Vn. The 52 kDa protein might contribute to the pathogenesis of biomaterial associated infections.

References

1. Kloos, W. E., and T. L. Bannerman. 1994. Update on clinical significance of coagulase-negative staphylococci. Clin Microbiol Rev. 7:117-40.

2. Herrmann, M., S. J. Suchard, L. A. Boxer, F. A. Waldvogel, and P. D. Lew. 1991. Thrombospondin binds to Staphylococcus aureus and promotes staphylococcal adberence to surfaces. Infect Immun. 59:279-88.

3. Lundberg, F., S. Schliamser, and Å. Ljungh. 1997. Vitronectin may mediate staphylococcal adhesion to polymer surfaces in perfusing human cerebrospinal fluid. J Med Microbiol. 46:285-96.

4. Nilsson, M., L. Frykberg, J. I. Flock, L. Pei, M. Lindberg, and B. Guss. 1998. A fibrinogen-binding protein of Staphylococcus epidermidis. Infect Immun. 66:2666-73.

5. Paulsson, M., Å. Ljungh, and T. Wadström. 1992. Rapid identification of fibronectin, vitronectin, laminin, and collagen cell surface binding proteins on coagulase-negative staphylococci by particle agglutination assays [see comments]. J Clin Microbiol. 30:2006-12.

6. Podack, E. R., B. Dahlback, and J. H. Griffin. 1986. Interaction of S-protein of complement with thrombin and antithrombin III during coagulation. Protection of thrombin by S-protein from antithrombin III inactivation. J Biol Chem. 261:7387-92.

7. Lundberg, F., D. Q. Li, D. Falkenback, T. Lea, P. Siesjo, S. Soderström, B. J. Kudryk, J. O. Tegenfeldt, S. Nomura, and Å. Ljungh. 1999. Presence of vitronectin and activated complement factor C9 on ventriculoperitoneal shunts and temporary, ventricular drainage catheters. J Neurosurg. 90:101-8.

8. Liang, O. D., J. I. Flock, and T. Wadström. 1995. Isolation and characterisation of a vitronectin-binding surface proteinfrom Staphylococciis aureus. Biochim Biophys Acta. 1250:110-6.

9. Liang, O. D., K. T. Preissner, and G. S. Chhatwal. 1997. The hemopexin-type repeats of human vitronectin are recognized by Streptococcus pyogenes. Biochem Biophys Res Commun. 234:445-9.

10. Ringnér, M., K. H. Valkonen, and T. Wadström. 1994. Binding of vitronectin and plasminogen to Helicobacterpylori. FEMS Immunol Med Microbiol. 9:29-34.

11. Shen, W., H. Steinruck, and Å. Ljungh. 1995. Expression of binding of plasminogen, thrombospondin, vitronectin, and fibrinogen, and adhesive properties by Escherichia coli strains isolated from patients with colonic diseases. Gut. 36:401-6.

12. Hrkal, Z., P. Cabart, and I. Kalousek. 1992. Isolation of human haemopexin in apoform by chromatography on S-Sepharose Fast Flow and Blue Sepharose CL-6B. Biomed Chromatogr. 6:212-4.

13. Vuento, M., and A. Vaheri. 1979. Purification of fibronectin from human plasma by affinity chromatography under non-denaturing conditions. Biochem J. 183:331-7.

14. Yatohgo, T., M. Izumi, H. Kashiwagi, and M. Hayashi. 1988. Novel purification of vitronectin from human plasma by heparin affinity chromatography. Cell Struct Funct. 13:281-92.

15. Paulsson, M., and T. Wadstrom. 1990. Vitronectin and type-I collagen binding by Staphylococcus aureus and coagulase-negative staphylococci. FEMS Microbiol Immunol. 2:55-62.

16. Laemmli, U. K. 1970. Cleavage of structural proteins during the assembly of the head of bacteriophage T4. Nature. 227:680-5.

17. Towbin, H., T. Staehelin, and J. Gordon. 1979. Electrophoretic transfer of proteins from polyacrylamide gels to nitrocellulose sheets: procedure and some applications. Proc Natl Acad Sci USA. 76:4350-4.

18. Rucheton, M., I. Stefas, I. Lamaury, J. Coste, J. Reynes, J. M. Lemaire, and H. Graafland. 1992. IgG autoantibodies against cellular p72 antigen crossing with (MLV) p15-gag antigen: presence in early HIV 1 infection, in HBV infection and in primary Gougerot-Sjogren. CR Acad Sci III. 314:533-8.

19. Bouchez-Mahiout, I., C. Doyen, and M. Lauriere. 1999. Accurate detection of both glycoproteins and total proteins on blots: control of side reactions occurring after periodate oxidation of proteins. Electrophoresis. 20:1412-7.

TABLE 1
Effect of protease and heat treatment of cells of S.
aureus V8 and S. epidermidis BD5703 on binding of
immobilized Vn.

| Treatment | V8 | | BD5703 | |
|---|---|---|---|---|
| Non-treated control | 100 | (9) | 100 | (4) |
| Heating^{a} | 98 | (5)^{b} | 91 | (4) |
| Pronase E | 49 | (6)^{d} | 18 | (4)^{d} |
| Protease K | 48 | (5)^{d} | 35 | (4)^{d} |
| Trypsin | 70 | (6)^{c} | 67 | (4)^{d} |

^{a}Bacterial cells heated at 1000° C. for 30 min and cooled down in ice bath
^{b}Relative percentage binding are presented as mean value (SEM). The experiments were done in triplicate and repeated twice
^{c}p<0.05 compared to non-treated control
^{d}p<0.01 compared to non-treated control

TABLE 2
Influence of different inhibitors on immobilized
Vn binding to S. epidermidis strain BD5703

| | Relative | |
|---|---|---|
| Inhibitors | binding percentage | |
| control (without inhibitor) | 100 | (4)^{c} |
| Soluble Vn (10 μg/ml)^{b} | 40 | (3)^{d} |
| Extract (5 μg/ml)^{a} | 4.33 | (2)^{d} |
| Extract treated by trypsin (5 μg/ml)^{a} | 98 | (3)^{e} |
| Heparin 1 (1 mg/ml)^{b} | 18 | (4)^{d} |
| Hyaluronic acid (1 mg/ml)^{b} | 41 | (3)^{d} |
| Heparin 2 (1 mg/ml)^{a} | 135 | (5)^{d} |
| Purified protein 1 (2 μg/ml)^{a} | 43 | (0.03)^{d} |
| Purified protein 2 (2 μg/ml)^{a} | 72 | (4)^{d} |
| Purified protein 3 (2 μg/ml)^{a} | 81 | (9)^{e} |

^{a}The components were preincubated with Vn coated wells for 2 hours at 37° C., and then bacteria were added.
^{b}The components were added at the same time as bacteria.
^{c}Data are presented as mean values (SEM). Triplicate samples were tested and repeated twice.
^{d}p < 0.01
^{e}No significant difference
Purified protein 1 is fraction 5 (60 and 52 kDa) eluted from Vn affinity column
Purified protein 2 is fraction 3 (21 kDa) eluted from Mono-S ® column
Purified protein 3 is fraction 11 (16 kDa) eluted from Mono-S ® column

TABLE 3
N-terminal of three vitronectin binding proteins of BD5703

| Molecular weight (kDa) | Purify method | N-terminal |
|---|---|---|
| 52 | Affinity column | TADPPADKTS^{a} |
| 21 | Mono-S ® column | MYAEYVNQLK |
| 16 | Mono-S ® column | GTAHSYWYKY |

^{a}weak signals

1. Protein isolated from Staphylococcus epidermidis having an approximate MW of 52 kD determined by SDS-PAGE and an N-terminal amino acid sequence of SEQ ID NO:1
Thr Ala Asp Pro Pro Ala Asp Lys Thr Ser
1 5 10
and antigenic determinant-containing fragments of the protein.

2. Protein or protein fragment according to claim 1 coupled to an inert carrier or matrix.

3. Recombinant DNA molecule coding for a protein or a protein fragment according to claim 1.

4. Vector selected from the group consisting of plasmids, phages or phagemides comprising a DNA molecule according to claim 3 or the corresponding RNA molecule.

5. Antibodies or antigen-binding peptides recognizing and specifically binding to a protein or protein fragment according to claim 1 or 2.

6. Use of a protein or protein fragment according to claim 1 or 2 as an immunizing component in the production of a vaccine against Staphylococcal infections.

7. Use of a vector according to claim 4 for the production of a vaccine against Staphylococcal infections.

8. Use of antibodies or antigen-binding peptides according to claim 5 for the production of a medicament for the passive immunization of a mammal against Staphylococcal infections.

9. Vaccine against Staphylococcal infections comprising as an immunizing component a protein or protein fragment according to claim 1 or 2.

10. Vaccine against Staphylococcal infections comprising a vector according to claim 4.

11. Medicament for the passive immunization of a mammal against Staphylococcal infections comprising antibodies or antigen-binding peptides according to claim 5.

12. Method of prophylactic and/or therapeutic treatment of Staphylococcal infections in a mammal comprising administration to said mammal of an immunologically effective amount of a vaccine according to any one of claims 9-11.
